(19) United States
(12) Patent Application Publication
(10) Pub. No.: US 2005/0186294 A1
(43) Pub. Date: Aug. 25, 2005

(54) Method of using a compound of menthol and L-arginine as a preparation for the topical delivery of vardenafil for the treatment of female sexual dysfunction

(76) Inventors: James M. Thompson, Cincinnati, OH (US); Justin R. Thompson, Cincinnati, OH (US); Ronald J. Thompson, Ft. Thomas, KY (US)

Correspondence Address: Donald N. Halgren, 35 Central Street, Manchester, MA, 01944, US

(21) Appl. No.: 11/105,228

(22) Filed: Apr. 13, 2005

Related U.S. Application Data
(63) Continuation-in-part of application No. 10/731,692, filed on Dec. 9, 2003.
Application No. 10/731,692 is a division of application No. 10/004,091, filed on Oct. 23, 2001, now Pat. No. 6,702,733.
Application No. 10/004,091 is a continuation of application No. 09/520,110, filed on Mar. 7, 2000, now Pat. No. 6,322,493.
Application No. 09/520,110 is a continuation-in-part of application No. 09/469,959, filed on Dec. 21, 1999, now abandoned.
Application No. 09/469,959 is a continuation-in-part of application No. 09/414,250, filed on Oct. 7, 1999, now Pat. No. 6,224,541.
Application No. 09/414,250 is a continuation-in-part of application No. 09/340,227, filed on Jul. 1, 1999, now Pat. No. 6,179,775.

(51) Int. Cl. A61K 31/198; A61M 35/00; A61K 35/78

(52) U.S. Cl. 424/739; 424/742; 424/745; 424/747; 514/565; 604/1

(57) ABSTRACT

A manual application of a topical clitoral sensitizing compound combination in an applicator, consisting essentially of L-arginine and a cooling agent comprised of Menthol and vardenafil, wherein the combination is applicable manually to the clitoris.

This invention relates to arrangements for the stimulation of females and more particularly to topical application of specialized stimulatory medicaments. This application is a continuation-in-part application of co-pending U.S. patent application Ser. No. 10/731,692 filed 9 Dec. 2003 which is a divisional application of U.S. patent application Ser. No. 10/004,091, filed 23 Oct. 2001, now U.S. Pat. No. 6,702,733 issued 9 Mar. 2004, which is a continuation of application Ser. No. 09/520,110, filed 7 Mar. 2000, now U.S. Pat. No. 6,322,493 which is a continuation-in-part of application Ser. No. 09/469,959 filed on 21 Dec. 1999, which is a continuation-in-part of application Ser. No. 09/414,250, filed on 7 Oct. 1999, now U.S. Pat. No. 6,224,541 which is a continuation-in-part of application Ser. No. 09/340,227, filed on 1 Jul. 1999, now U.S. Pat. No. 6,179,775 each of which are incorporated herein by reference in their entirety.

### FIELD OF THE INVENTION

### PRIOR ART

U.S. Pat. Nos. 6,322,493 and 6,702,733 describe the method of inducing the nitric oxide synthase pathway to produce nitric oxide and cyclic GMP (cGMP) by the topical application of menthol and L-arginine to the female clitoris. This menthol and L-arginine combination acts to initially dilate the vasculature of the mucosa overlying the corpus cavemosa of the clitoris by action of menthol, and, secondarly, to actively dilate the plexes of the spongiform corpus cavemosa to engorge it with blood to create a clitoral erection. This is accomplished by the L-arginine inducing the production of nitric oxide via the specific nitric oxide isoform, inducible nitric oxide synthase (iNOS).

U.S. patent application Ser. No. 10/803,148, applied Mar. 17, 2004, explains the added benefit of using a 5-Phosphodiesterase Inhibitor in combination with menthol and L-arginine for the treatment for female sexual dysfunction. Increasing intracellular concentrations of L-arginine leads to heightened production of nitric oxide and cyclic GMP, which results in vasodilatation of the corpus cavemosa via the nitric oxide synthase pathway. The 5-Phosphodiesterase inhibitor blocks the degradation of cGMP and promotes increased cellular levels of cGMP in the corpus cavemosa, again resulting in vasodilatation of the corpus cavemosa.

In U.S. Pat. No. 6,338,862, “Composition and method of use in treating sexual dysfunction using cGMP-specific PDE-5 inhibitors,” Niazi writes of the benefits of using sildenafil in combination with L-arginine for the treatment of sexual dysfunction. Applied Mar. 26, 2001, and issued Jan. 15, 2002, U.S. Pat. No. 6,388,862 describes the use of a 5-PDE inhibitor but does not reference a specific vehicle needed to carry a large molecule like sildenafil across the mucous membrane into the corpus cavernosa. In U.S. Pat. No. 6,403,658, titled “Genital Vasodilitation,” Niazi references sildenafil as a smooth muscle cell relaxant for increasing blood flow to the genitals.

Issued Oct. 22, 2002, U.S. Pat. No. 6,469,016, “Treatment of Female Sexual Dysfunction using Phosphodiesterase Inhibitors,” writes of the potential benefit of using phosphodiesterase inhibitors for the treatment of female sexual dysfunction. This patent references sildenafil and tadalafil as two phosphodiesterase type-5 specific inhibitors that could be used.

U.S. Pat. No. 6,472,434, “Method for minimizing Excess Collagen Deposition” was issued Oct. 29, 2002. This patent describes the use of a 5-PDE inhibitor and a PGE1 (prostaglandin E1) to treat dysparunia (pain during sex that is experienced in 40% of women) due to inadequate lubrication and for the minimization of collagen misdeposition due to hypoxia.

### BACKGROUND

Vardenafil

Since the introduction of oral 5-Phosphodiesterase Inhibitor medications for erectile dysfunction in 1998, compounding pharmacists and physicians have attempted to develop a topical preparation of a 5 Phosphodiesterase Inhibitor to treat women with female sexual dysfunction, but this has proven less than effective because of the size of the 5-PDE Inhibitor molecule. Even when topically applied to the mucous membrane, which is more absorptive than hairy skin, the size of the vardenafil molecule, a 5 phosphodiesterase inhibitor, with a large molecular weight, 579.2 g/Mol, requires the aid of a vehicle, or absorbing agent, to allow absorption into the corpus cavernosa of the clitoris.

Vardenafil is a potent 5 Phosphodiesterase Inhibitor, a molecule that blocks the degradation of cGMP by specifically binding to the catalytic site of 5 Phosphodiesterase molecules. Blount et al, in “Binding of Titrated Sildenafil, Tadalafil, or Vardenafil to the Phosphodiesterase-5 Catalytic Site Displays Potency, Specificity, Heterogeneity, and cGMP Stimulation,” found that the “absolute potency values were similar for each inhibitor, and the relative potencies were vardenafil>tadalafil>sildenafil.” Vardenafil, sildenafil, and tadalafil are a group of inhibitors that bind restrictively to the catalytic domain of 5 Phosphodiesterase; “all three inhibitors also bind to the same catalytic site.” Blount's team also reported IC₅₀ and K_{D} (binding isotherm) values for each of the three inhibitors and were found to be 41:2:1 and 13:2:1 for vardenafil, tadalifil and sildenafil, respectively. The higher potency of vardenafil is due to differences in its double ring molecular structure. Blount also reported that increases in temperature closer to the normal human body temperature led to “increased vardenafil binding in the presence of cGMP,” for 30 degrees Celsius versus 4 degrees Celsius. In vitro testing performed by Blount's group has also confirmed 100% inhibition of 5 Phosphodiesterase activity by the vardenafil molecule, where at similar Molar concentrations sildenafil and tadalifil were only 85% and 75% respectively.

Menthol

The menthol component acts as an immediate vasodilator of the vessels in the mucous membrane of the clitoral tissues and functions as a vehicle to facilitate the transport of the vardenafil and L-arginine across the mucous membrane. Menthol has been shown clinically to increase membrane permeability by 2-4 times and decrease Tₘ values for larger molecular weight molecules such as Testosterone, molecular weight 288.42 g/Mol (Kaplan Frischoff et al). This increased permeability from the Menthol component allows both the L-arginine and vardenafil to cross the membrane and enter the corpus cavemosa.

L-Arginine

The L-arginine component of the menthol and L-arginine combination functions to induce the nitric oxide pathway as described in U.S. Pat. No. 6,702,733, incorporated by reference as stated hereinabove. L-arginine is a semi-essential amino acid that is a component of a normal diet and stored in cellular tissues in minute amounts. By presenting L-arginine in abundant amounts directly to the corpus cavemosa, dietary intake of L-arginine is unnecessary for normal sexual responsiveness.

L-arginine concentrations have been well studied and characterized in humans after the discovery of the Nitric Oxide Synthase by a number of clinical researchers. The basal nitric oxide has been found to be 100 nMol/ml (Kamada) and 85 nMol/ml (Bode-Boger) in different studies with overlapping standard errors. After infusion of 30 g and 6 g of L-arginine levels in the blood rise to 6223 nMol/ml and 822 nM/ml, which correspond to a rise in detectable cGMP excretion rates in urine. After oral arginine supplementation (6 g), the blood concentration rose to only 310 nMol/ml, and the increases in detectable cGMP were not statistically significant. Bode-Boger et al published “L-arginine-induced vasodilation in healthy humans: pharmacokinetic-pharmacodynamic relationship” in the British Journal of Clinical Pharmacology, 1998. In the article, Bode-Boger describes the relationship between L-arginine and nitric oxide,
“L-arginine is the physiological precursor of nitric oxide (NO), an important mediator of vasodilation and inhibition of platelet aggregation via increased formation of cyclic GMP. NO is synthesized in endothelial cells from the terminal guanidine nitrogen of L-arginine by the activity of the endothelial, calcium-dependent isoform of NO synthase.”

Okada and Asakawa, in their article “Allosteric Activation of cGMP-Specific, cGMP-Binding Phosphodiesterase (PDE5) by cGMP,” concluded that the “effects of competitive PDE inhibitors are influenced by the concentrations of the substrate cyclic nucleotides.” Moreover, the generation of the substrate, cyclic GMP, is regulated by the signal transduction system; nitric oxide synthase pathway. This system is a self-regulating negative feedback loop, with multiple cellular mechanisms to inhibit excess NO and cGMP, which effect vasodilitation. The conversion of L-arginine to NO, then cGMP, which is then degraded by the 5 Phosphodiesterase occurs during a very small timeframe, in which, as cGMP levels are increased, the 5PDE becomes increasingly more effective in catalysis of cGMP, as well as allosteric binding to decrease plasma cGMP, as well as a number of other molecular actions as described by Corbin et al, 2003. If nNos (see below) signaling is not present, the arginine concentration must pass a “threshold” to induce the negative feedback Nitric Oxide Synthase Pathway to produce increased levels of cGMP that have dilatory effect. With the relatively small blood volume circulating in the clitoris corpus cavemosa before arousal, and then slightly increased blood volume sequestered during a clitoral erection (fully aroused female), the effective L-arginine present in the female clitoral tissues is very small and would be converted very quickly to NO and cGMP and readily degraded by 5 Phosphodiesterase.

Dual Actions

A synergistic effect of vasodilatation of the clitoral corpus cavemosa can be expected from the dual mechanisms of the menthol/L-arginine induction and fueling of cyclic GMP production, and then by the vardenafil inhibition of cyclic GMP degradation. In both of the above mechanisms, nitric oxide (NO), is the intermediate signaler, or messenger, that evokes cyclic GMP production. NO is a vasodilator itself, but has a very short half life. Cyclic GMP is a potent vasodilator but is readily degraded as described above. The duel mechanism of action of the combination of menthol/L-arginine and vardenafil can be accurately described as different front-end and back-end mechanisms of action. Both the front-end mechanism of action and the back-end mechanism of action result in a sustained increase over a threshold in effective cyclic GMP, and therefore sustained increased vasodilatation of the corpus cavemosa of the clitoris.

The Nitric Oxide Synthase Pathway is:

The front-end mechanism of action is the induction of the nitric oxide synthase enzyme, iNOS. The back-end mechanism of action is the PDE-5 inhibition by the action of the potent specific PDE-5 inhibitor, vardenafil. By combining the front-end and the back-end mechanisms of action together, multiple benefits can not only be expected, but confirmed by clinical trials using the topical menthol/L-arginine and vardenafil to increase blood flow to the clitoris and therefore increase in a woman's sexual responsiveness. One benefit of the combination of the above described topical combination would be to use a lower dosage or concentration of each component and to still be able to achieve clinical effectiveness. Working on both the front-end and the back-end of the same pathway will allow the combination to be greater than the sum of its parts. A second benefit of the above described topical combination would be an increased effectiveness of vasodilatation and sexual stimulation, helping to create a statistical difference from a topical placebo and proving efficacy. This is important because all of the measures of effectiveness over placebo are subjectively reported by women in clinical studies, and each woman's sexual responsiveness is a multi-factional problem.

A third, and somewhat obscure rationale, is the utilization of the synergistic duel mechanisms of the action of the menthol/L-arginine and the vardenafil to increase the effectiveness of the topical combination with each successive use. McCullough et al studied the effectiveness of oral sildenafil in men. Titled “Intercourse success rates with sildenafil citrate,” McCullough found that sildenafil was only 54% effective after its first use to create a penile erection. By the eighth use, the effectiveness of sildenafil had increased to 86%. This effect, increased effectiveness with increased usage, is biochemically and physiologically explained by the increasing concentrations of the actual nitric oxide synthase enzyme with successive uses of any of the PDE-5 inhibitors. In fact, decreasing sexual responsiveness in both men and women has been attributed to age related decreases in nitric oxide synthase enzyme concentrations

By utilizing the clinically proven potent duel mechanisms of the nitric oxide induction and inhibition of cyclic GMP degradation, the nitric oxide synthase enzyme concentrations can be expected to increase dramatically; even between the first and second use of the topical menthol/L-arginine/vardenafil combination. This dramatic increase in nitric oxide synthase enzyme concentrations, counter acting the effects of aging, would be expected to maximize clitoral corpus cavemosa vasodilatation and maximize women's sexual responsiveness even with the second or third use of the topical combination of menthol/L-arginine/vardenafil.

The Isoforms of The Nitric Oxide Synthase Enzyme

Three isoforms of the nitric oxide synthase enzyme have been identified and extensively reported upon in the medical literature over the past several years. The isoforms are named for the tissue type where they have the largest concentration. The three isoforms are nNOS, neuronal, eNOS, endothelial (the cells lining blood vessels), and iNOS, inducible nitric oxide synthase. The iNOS is found in all tissues except nerve tissue and endothelial tissue. Since the nerve tissue and the endothelial tissue only represent ten to fifteen percent of the total tissue, iNOS accounts for eighty five to ninety percent of all the NOS enzyme present in a specific tissue. The descriptive name of inducible NOS enzyme is from researchers who identified iNOS. iNOS is named inducible because of its unique ability to be activated or induced to produce nitric oxide from a high concentration of L-arginine, the only substrate for the nitric oxide synthase pathway.

The isoforms of NOS are present in all tissues, but much more concentrated in specific target tissue that rely upon the NOS and cyclic-GMP to produce smooth muscle relaxation and vasodilatation of that specific tissue to accomplish that tissue's function. NOS isoforms are concentrated in vascular tissue, for blood vessel dilatation, pulmonary tissue for air way dilatation, but most concentrated in the corpus cavemosa tissues of the penis and clitoris. Active smooth muscle relaxation and dilatation of the corpus cavernosa, and the passive engorgement with blood, is the physiological basis of both the penile and clitoral erection. These erections are established by the activation of the nitric oxide synthase isoforms, predominantly the iNOS, to produce nitric oxide, cyclic GMP and effect potent smooth muscle cell relaxation. A report in the December 2002 Experimental Biological Medicine by Park et al, titled “Nitric oxide-cyclic GMP signaling pathway in the regulation of rabbit clitoral cavemosa tone” concluded that the degree of erection of the corpus cavemosa tissue of the rabbit clitoral tissue is directly related to the activity level of the nitric oxide synthase isoenzymes. A more specific medical reference relates the in-vitro pathway of vardenafil to accomplish vasodilatation and corpus cavemosa smooth muscle dilatation to effect a clitoral or penile erection. Dr. J S Kalsi et al in the February 2003 Journal of Urology reported that vardenafil resulted in concentration dependant relaxation of human and rabbit cavernosum in “BA741-2272, a novel nitric oxide independent soluble guanglate cyclase activator, relaxes human and rabbit corpus cavernosum in vitro.”

The actions of vasodilatation and smooth muscle relaxation of the corpus cavernosa are actually one in the same. The corpus cavernosa, in its resting non-excited state, can be thought of as swiss cheese with very small holes. The holes represent the veneous plexes of the corpus cavernosa. When the smooth muscle of the tissue surrounding the venous plexes relaxes, the venous plexes dilate, to allow blood to fill the plexus. This can be envisioned as swiss cheese with very large holes. In fact, the corpus cavernosa in its' erect state is ninety percent blood and only ten percent smooth muscle. The smooth muscle relaxation of the corpus cavernosa, and the resultant vasodilatation of venous plexus, is accomplished by the nitric oxide synthase production of nitric oxide and cyclic GMP by all of the NOS isoforms, but predominantly, the inducible NOS, the predominant isoenzyme in clitoral or penile corpus cavemosa.

2004 Vardenafil 1%/CSC vs. CSC Clinical Trial

Each woman was asked to use CSC at least 3 times and then use the Vardenafil 1%/CSC at least 3 times before reporting results

Each woman was forewarned about possible burning if her estrogen status was low or absent (establishing expectations as every physician does when giving a patient a prescription)

Population Demographics:

| Age Group | N= |
|---|---|
| 20-34 | 12 |
| (on BCP's) | |
| 30-39 | 13 |
| 40-49 | 11 |
| 50-59 | 8 |
| Total Women | 44 |

Results:

Percentage of Participants (n/N) Reporting an Increase in Rating over CSC

TABLE 2A*
Vardenafil 1%/CSC Percentage Rating
Increase Over CSC (0-5); 2.5 = CSC

| | | | Speed | Ease of | | |
|---|---|---|---|---|---|---|
| | | Arousal | to | Achieving | Orgasm | Multiple |
| | N= | Lubrication | Arousal | Orgasm | Intensity | Orgasms |
| 22-34 | 12 | 91.6% | 91.6% | 100% | 100% | 91.6% |
| (BCP) | | | | | | |
| 30-39 | 13 | 100% | 100% | 100% | 100% | 84.6% |
| 40-49 | 11 | 100% | 100% | 100% | 100% | 90.9% |
| 50-59 | 8 | 100% | 100% | 100% | 100% | 62.5% |
| Average | 44 | 97.7% | 97.7% | 100% | 100% | 84.1% |

| | | | Ease of | | |
|---|---|---|---|---|---|
| | Arousal | Speed to | Achieving | Orgasm | Multiple |
| | Lubrication | Arousal | Orgasm | Intensity | Orgasms |
| 22-34 | 40.0% | 36.7% | 80.0% | 86.7% | 23.3% |
| (BCP) | | | | | |
| 30-39 | 30.9% | 35.1% | 62.9% | 67.9% | 16.3% |
| 40-49 | 45.5% | 45.5% | 81.8% | 78.2% | 27.3% |
| 50-59 | 50.0% | 55.0% | 65.0% | 75.0% | 10.0% |
| Overall | 43.6% | 45.4% | 75.5%* | 77.3%* | 21.8% |
| Avg | | | | | |

*Esentially, all participants reported the Vardenafil 1%/CSC was a better product than the CSC alone to help achieve more intense orgasms

Combined Data: Added “Value” of Vardenafil 1% in the CSC
Formula:
(1+% Rating Inc over CSC)×% Participants Reporting Increase of V1 % CSC

TABLE 3
X % Increase over Pretest

| | | Increased | | Increased | Increased |
|---|---|---|---|---|---|
| | Lubrication Speed | Orgasm | Speed to Achieve | Orgasm | Multiple |
| | and Quality | Percentage | Orgasm | Intensity | Orgasms |
| 20-36 | 1.40* .916* | 1.80* 1.0* | 1.367* .916* | 1.867* 1.00* | N/A |
| (BCP) | 19% | 30.6% | 31.9% | 37.5% | |
| | 24.4% | 55.1% | 39.9% | 70.0% | |
| 20-29 | 1.40* .916* | 1.80* 1.00* | 1.367* .916* | 1.867* 1.00* | N/A |
| | 15% | 33.5% | 26.9% | 33.3% | |
| | 19.2% | 60.3% | 33.7% | 62.2% | |
| 30-39 | 1.309* 1.00* | 1.629* 1.00* | 1.351* 1.00* | 1.679* 1.00* | N/A |
| | 23% | 40.0% | 35.4% | 48.1% | |
| | 30.1% | 65.2% | 47.8% | 80.8% | |
| 40+ | 1.455* 1.00* | 1.818* 100* | 1.458* 1.00* | 1.782* 1.00* | N/A |
| | 18% | 25.3% | 35.4% | 48.1% | |
| | 26.2% | 46.0% | 51.6% | 85.7% | |
| Overall | 25.1% | 56.7% | 43.3% | 74.7% | N/A |
| Avg | | | | | |

Analysis

1. Lubrication speed and quality is not improved by vardenafil 1%/CSC over CSC

2. Increased orgasm percentage and increased orgasm intensity are very significantly improved with the vardenafil 1%/CSC compared to the CSC alone

Since the very definition of sexual satisfaction is the ability to achieve a meaningful orgasm, and all of the validated instruments measure sexual satisfaction for FSD, the vardenafil 1%/CSC can be confidently predicted to establish efficacy over placebo with statistically significant results.

### DESCRIPTION OF THE PRESENT INVENTION

The present invention relates to a manually applicable topical preparation comprising a compound of menthol or a menthol substitute, and L-arginine, and vardenafil as that topical compound. This manually applicable compound is to be manually applied directly to a woman's clitoris, for the treatment of female sexual dysfunction. Vardenafil in one preferred embodiment may preferably comprise less than 10% of that topical compound.

A first preferred embodiment of a menthol substitute may be peppermint oil. A second preferred embodiment of a menthol substitute may be cornmint oil. A third preferred embodiment of a menthol substitute may be Eucalyptus oil. A fourth preferred embodiment of a menthol substitute may be Citronella oil. A fifth preferred embodiment of a menthol substitute may be Camphor oil. A sixth preferred embodiment of a menthol substitute may be Cinnamon oil. A seventh preferred embodiment of menthol would include menthol analogs and derivatives including: (+)-neo-Menthol, Menthone, (+)-iso-Menthone, Menthyl acetate, Menthyl isovalerate, (−)-Menthyl lactate, para-Menth-1-en-3-ol, Piperitone, (−)-Menthol ethylene glycol carbonate, (−)Menthol 1- and 2-propylene glycol carbonate, (−)-Mentholel 1,2-glycerol ketal, (+)-Menthonel 1,2-glycerol ketal, and mono-Methyl succinate.

The invention thus comprises a manual application of a topical clitoral sensitizing combination consisting essentially of L-arginine and a cooling agent comprised of menthol and vardenafil wherein the combination is applicable manually to the clitoris.

The cooling agent may in various preferred embodiments include peppermint oil. The cooling agent may include cornmint oil. The cooling agent may include Eucalypus oil. The cooling agent may include Citronella oil. The cooling agent may include Camphor oil. The cooling agent may include Cinnamon oil. The cooling agent may essentially comprise peppermint oil. The cooling agent may essentially comprise cornmint oil. The cooling agent may essentially comprise Eucalyptus oil. The cooling agent may essentially comprise Citronella oil. The cooling agent may essentially comprise Camphor oil. The cooling agent essentially may comprise Cinnamon oil. The combination may contain less than 10% L-arginine. The combination may contain less than 10% menthol and the combination may contain less than 10% L-arginine and also the combination may contain less than 10% vardenafil.

1. A manual application from an applicator of a topical clitoral sensitizing combination consisting essentially of L-arginine and a cooling agent comprised of Menthol and vardenafil, wherein said combination is applicable manually to the clitoris.

2. The topical sensitizing combination as recited in claim 1, wherein said cooling agent includes Peppermint oil.

3. The topical sensitizing combination as recited in claim 1, wherein said cooling agent includes Cornmint oil.

4. The topical sensitizing combination as recited in claim 1, wherein said cooling agent includes Eucalypus oil.

5. The topical sensitizing combination as recited in claim 1, wherein said cooling agent includes Citronella oil.

6. The topical sensitizing combination as recited in claim 1, wherein said cooling agent includes Camphor oil.

7. The topical sensitizing combination as recited in claim 1, wherein said cooling agent includes Cinnamon oil.

8. The topical sensitizing combination as recited in claim 1, wherein said cooling agent essentially comprises Peppermint oil.

9. The topical sensitizing combination as recited in claim 1, wherein said cooling agent essentially comprises Cornmint oil.

10. The topical sensitizing combination as recited in claim 1, wherein said cooling agent essentially comprises Eucalyptus oil.

11. The topical sensitizing combination as recited in claim 1, wherein said cooling agent essentially comprises Citronella oil.

12. The topical sensitizing combination as recited in claim 1, wherein said cooling agent essentially comprises Camphor oil.

13. The topical sensitizing combination as recited in claim 1, wherein said cooling agent essentially comprises Cinnamon oil.

14. The topical sensitizing combination as recited in claim 1, including one or more Menthol Analog from the group consisting of:
a. (+)-neo-Menthol
b. Menthone
c. (+)-iso-Menthone
d. Menthyl acetate
e. Menthyl isovalerate
f. (−)-Menthyl lactate
g. para-menth-1-en-3ol
h. Piperitone
i. (−)-Menthol ethylene glycol carbonate
j. (−)-Menthol 1-and 2-propylene glycol carbonate
k. (−)-Menthone 1,2-glycerol ketal
l. (+)-Menthone 1,2-glycerol ketal
m. mono-Menthyl succinate

15. The topical sensitizing combination as recited in claim 1, wherein said combination contains less than 10% L-arginine.

16. The topical sensitizing combination as recited in claim 1, wherein said combination contains less than 10% menthol and said combination contains less than 10% L-arginine and said combination contains less than 10% vardenafil.

17. A Method creating maximum vasodilatation and stimulation of the female clitoris by the steps of:
applying a topical application of a compound utilizing the duel actions of nitric oxide and cyclic GMP induced production, via the actions of menthol and L-arginine, and the inhibition of the cyclic GMP degradation, by vardenafil in said compound, to create said vasodilatation and stimulation of the female clitoris.

18. The method of achieving effective vasodilatation and maximum stimulation of the female clitoris, as recited in claim 17, by the step of:
decreasing the dosage of menthol/L-arginine and/or vardenafil when topically applied to the female clitoris, to achieve said effective vasodilatation and maximum stimulation of the female clitoris.

19. A method to stimulate the female clitoris by the step of:
utilizing menthol as a vehicle for vardenafil transport across the mucous membrane into the clitoral corpus cavernosa.

20. A method to stimulate the female clitoris by the step of:
utilizing L-arginine to provide a substrate for the production of cyclic GMP to therefore maximize the vasodilatation of the clitoral corpus cavernosa by the actions of vardenafil to inhibit the enzymatic degradation of that cyclic GMP.
